(19) United States
(12) Patent Application Publication
(10) Pub. No.: US 2003/0049600 A1
(43) Pub. Date: Mar. 13, 2003

(54) STEALTH VIRUS DETECTION IN THE CHRONIC FATIGUE SYNDROME

(76) Inventor: WILLIAM J. MARTIN, SOUTH PASADENA, CA (US)

Correspondence Address: LYON & LYON LLP, 633 WEST FIFTH STREET, SUITE 4700, LOS ANGELES, CA, 90071, US

(21) Appl. No.: 08/463,186

(22) Filed: Jun. 5, 1995

Related U.S. Application Data
(63) Continuation-in-part of application No. 08/157,811, filed on Nov. 23, 1993, now Pat. No. 5,756,281.
Application No. 08/157,811 is a continuation-in-part of application No. 07/887,502, filed on May 22, 1992, now abandoned.
Application No. 07/887,502 is a continuation-in-part of application No. 07/704,814, filed on May 23, 1991, now abandoned.
Application No. 07/887,502 is a continuation-in-part of application No. 07/763,039, filed on Sep. 20, 1991, now abandoned.

(51) Int. Cl. C12Q 1/70; C12N 5/08

(52) U.S. Cl. 435/5; 435/366

(57) ABSTRACT

A method of detecting a stealth virus is provided by culturing a sample under conditions in which any stealth virus in the sample is able to induce a cytopathic effect. A method for culturing a virus is also provided by (a) cocentrifuging a sample of said virus with a permissive cell line of indicator cells; (b) inoculating the cell mixture into culture vessels; (c) adding viral enhancing medium to the culture; and (d) detecting in vitro a cytopathic effect in the permissive cell line.

### RELATED APPLICATIONS

This application is a continuation-in-part of co-pending U.S. patent application Ser. No. 08/157,811, filed Nov. 23, 1993, which is a continuation-in-part of application Ser. No. 07/887,502, filed May 22, 1992 entitled “Stealth Virus Detection in the Chronic Fatigue Syndrome” which is a continuation-in-part application of U.S. patent application Ser. No. 07/704,814, filed May 23, 1991; and Ser. No. 07/763,039, filed Sep. 20, 1991 entitled “Spumavirus detection in the chronic fatigue syndrome”. These prior submissions, including any drawings, are incorporated by reference in their entirety herein.

### 1. FIELD OF INVENTION

The present invention relates generally to agents that neutralize viral-associated toxins.

### 2. BACKGROUND OF THE INVENTION

A. Chronic Fatigue Syndrome

Palca, Science, 249:1240-1241 (1990) and Palca, Science, 254:1726-1728 (1991) describe attempts to identify a causative agent for chronic fatigue syndrome.

DeFreitas et al., Chemical Abstracts, 114: Abstract No. 205331c (1991) describes retroviral sequences related to human T lymphotropic virus type 2 in patients with chronic fatigue immune dysfunction syndrome.

Gupta et al., Scandinavian Journal of Immunology, 33:319-327 (1991) describes a comprehensive immunological analysis of chronic fatigue syndrome. The analysis of cell mediated and antibody mediated immunity was performed in 20 patients with chronic fatigue syndrome and 20 age and sex matched healthy controls.

B. Culture Techniques

Werner, Lancet, II:258-259 (1979) describes the isolation of foamy viruses from patients with de Quervain Thyroiditis and the detection of a cytopathic effect.

DiLuca et al., Virology, 175:199-210 (1990) describes the replication of viral and cellular DNA in human herpesvirus 6-infected cells and the use of medium RPMI 1640 containing 10% fetal calf serum.

Ablashi et al., International Journal of Cancer, 42:787-791 (1988) describes the use of human hematopoietic cell lines for the propagation of HBLV (human herpesvirus 6) in RPMI 1640 supplemented with 10% FBS and antibiotics.

Rethwilm et al., Nucleic Acids Research, 18:733-738 (1990) describes an infectious molecular clone (pHSRV) of the human spumaretrovirus (HSRV). pHSRV derived virus produced foamy virus typical cytopathic effects in susceptible cultures.

### SUMMARY OF THE INVENTION

The present invention provides a method for detecting a stealth virus by culturing a sample under conditions that induce a cytopathic effect. Several culture conditions are sufficient to induce a cytopathic effect in a sample containing a stealth virus. These culture conditions include replacing the culture medium every 24 to 72 hours, adding 5% to 10% fetal calf serum to the culture medium, using serum free medium X Vivo-15, using preculture centrifugation and adding viral enhancing medium to the culture.

Agents that neutralize stealth viral associated toxins are described in commonly owned international application, publication No. WO 92/20787, published Nov. 26, 1992, incorporated herein by reference in its entirety, including any drawings.

In one aspect the present invention provides a kit for in vitro culture of stealth virus comprising an agent of that neutralizes stealth virus-associated toxic activity.

In preferred embodiments the neutralizing agent is an antibody to the stealth virus-associated toxin or epione and the kit may also contain a container and/or a label and/or other conventional kit components well known to those skilled in the art.

In one aspect, a method of detecting a stealth virus is provided by culturing a sample under conditions in which any stealth virus in the sample is able to induce a cytopathic effect.

The term “stealth virus” refers to a virus having all of the following characteristics: (a) the ability to induce a cytopathic effect in fibroblastic cultures, including primary kidney cell cultures, which is characterized by the production of enlarged, foamy appearing cells, including cell syncytia; (b) the ability to produce inhibitory compounds capable of suppressing viral growth; (c) the ability to grow in cells from a plurality of species; (d) the ability of viral-specific typing antisera to distinguish stealth viral infected cells from cells infected with human cytomegalovirus, herpes simplex virus, human herpesvirus-6, varicella zoster virus, Epstein-Barr virus, adenoviruses or human T lymphotropic virus (HTLV); (e) the ability of virus specific nucleic acid probes to distinguish stealth viral infected cells from cells infected with human cytomegalovirus, herpes simplex virus, human herpesvirus-6, varicella zoster virus, Epstein-Barr virus, adenoviruses or HTLV, using stringent hybridization conditions; and (f) the ability to cause disease in an immunocompetent host without evoking an inflammatory response throughout the infected tissues.

The term “cytopathic effect” (CPE) refers to the appearance of rounded, slightly enlarged, refractile cells throughout the culture. In some cultures the CPE progresses to very prominent collections of tightly packed, enlarged, foamy-cell appearing cells, with clearly defined cell syncytia and evidence of considerable cell destruction. Stages between the spindle shape of the normal fibroblasts and the rounded appearance of affected cells can be seen. Several inclusions, consistent with vacuoles, can been seen within the cytoplasm. As their numbers increase, affected cells form several tightly adherent clumps with indistinct cell boundaries. The affected cells continue to proliferate and scatter away from the cell clumps.

In preferred embodiments the presence of the stealth virus is detected by (1) inoculating a permissive cell line with a sample, and (2) detecting in vitro a CPE in the permissive cell line; the cell line is maintained in culture medium that is replaced every 24 to 72 hours; the cell line is an insect cell line e.g., the cell line is derived from a plurality of species and may even include Spodoptera fruiperdo derived ovarian cell line Sf9; and the sample is from a patient suspected of having chronic fatigue syndrome (CFS) based upon other recognized criteria well known to physicians in the art.

The term “chronic fatigue syndrome” (CFS) refers to an illness whose major characteristic is an unexplained fatigue lasting beyond 6 months which results in greater than 50% reduction in an individual's normal level of activity (Holmes et al., “Chronic fatigue syndrome: A working case definition,” Ann. Intern. Med ., 108:387-389 (1988); Holmes, “Defining the chronic fatigue syndrome”, Rev. Inf. Dis., 13 (Suppl. I):S53-5 (1991); Shafan, “The chronic fatigue syndrome” Am. J. Med., 90:731-738 (1991)). To establish a clinical diagnosis, the patients should show evidence of suffering at least eight of the following minor symptoms: fever, sore throat, myalgia, muscle weakness (which may be exacerbated by exercise), arthralgia, lymphadenopathy, sleep disturbance, headaches, acute or subacute onset, and neuropsychological symptoms. The neuropsychological symptoms include a difficulty in thinking, dysnomia, confusion, forgetfulness, irritability, depression, photophobia and transient visual scotomata.

A cell is “permissive” if a particular virus causes a productive infection in it. A productive infection is a viral infection of a cell that produces progeny with the vegetative or lytic cycle. A productive infection by a stealth virus is characterized by the appearance of a cytopathic effect. Using appropriate conditions, stealth viruses infect and propagate in many mammalian cells in vitro, especially cultures of fibroblastic cells including primary kidney cell cultures, and other cell lines of epidermal, mesodermal, neuroectodermal and lymphoid origin, e.g., glial cells, myoblasts, etc. The distinctive CPE is, however, readily observed in primary fibroblast cultures. At present, therefore, these are the preferred cell lines to isolate the virus from clinical and animal samples. Suitable cells include human MRC-5 lung fibroblasts, primary human foreskin fibroblasts MRHF and rhesus monkey kidney RMK cells. These well known cell lines are available commercially (e.g., from Whittaker Bioproducts, Walkersville, Md.). It should be noted that not all cell lines are permissive for growth and/or for the expression of a cytopathic effect (CPE).

Furthermore, the CPE developing within a culture can be non-progressive or may actually regress. The lack of progression of the CPE and the regression of CPE are related to the presence of compounds within the culture supernatants. These compounds can be categorized as having (i) a general cellular toxicity effect in which the cultured cells exhibit a reduction in metabolic activity with an overall reduced vitality. Cells so affected do not actually die and, in fact, can outlast the period in which control cultures will have exhausted their media and perish. Cells exposed to stealth virus associated toxins do not readily support the expression of the CPE associated with stealth viruses. The second category of compounds that can be identified in certain stealth viral cultures are those that can inhibit stealth virus growth in the absence of observable toxicity. Such viral inhibitory factors are found in culture supernatants from certain cell lines infected with stealth viruses. These compounds are referred to as inhibitors. They may show activity not only on stealth viruses but also on other viruses including HIV.

In other embodiments, the toxic compounds present in a stealth virus infected individual are neutralized in vivo as a potential therapy for suppressing the symptoms of a stealth virus infection.

In other embodiments, the inhibitory compounds are utilized as a potential therapy for suppressing the in vivo growth of stealth viruses or other viruses susceptible to the viral inhibitory effects.

In another aspect a method of detecting a stealth virus is provided by: (1) inoculating a permissive cell line with a sample; (2) neutralizing stealth virus-associated toxic activity; and (3) detecting in vitro a cytopathic effect in the permissive cell line.

The term “stealth virus-associated toxin” or “toxin” refers to the molecular entity (or entities) that mediates the toxic activity observed in stealth virus cultures in vitro, and which appears capable of suppressing stealth virus growth. Cultures with toxin are often more difficult to infect with other stealth viral isolates. The toxic activity is detectable in serum and cerebrospinal fluid from stealth virus infected patients and may mediate certain symptoms associated with the patient's illness.

In preferred embodiments the toxic activity is neutralized by frequently replacing the culture medium, using medium in which the production of the toxin is minimized, or by adding a neutralizing agent to the culture; the neutralizing agent is fetal calf serum or retinoic acid, an antibody specific for the toxin, or a chemical or virus derived agent capable of competing with or reversing the toxic activity; and the sample is derived from a human, animal, food, or other environmental substance or object.

The term “neutralized” refers to any amount of inhibition or decrease. Thus, by neutralizing the toxic activity, one actually increases the detection of stealth virus. The term “replacing” refers to removing old culture medium and transferring new culture medium into the sample being tested to a new culture medium.

Stealth viruses may be isolated from a sample of blood or other biological samples including surgical and fine needle aspiration tissue biopsies, post mortem organ biopsies, throat swabs and saliva, urine, cerebrospinal fluid (CSF), other body fluids, blood and blood products intended for transfusion or for in vitro uses, vaccines, foods, and from the environment.

In another aspect, a method for detecting a stealth-virus is provided by: (1) inoculating a permissive cell line with a sample of the virus in a culture; (2) adding serum free medium to the culture; and (3) detecting in vitro the presence of a CPE in the permissive cell line.

In preferred embodiments, the serum free medium is medium X Vivo-15 (BioWhittaker, Inc., Walkersville, Md.). X Vivo-15 is a Iscove's modified Delbecco's medium with the addition of human albumin, human insulin and human transferrin. Iscove's modification of Delbecco's medium is intended to support high density cell growth and has additional glucose, salts and vitamins compared to Delbecco's medium (Iscove and Melchers, J. Experimental Medicine, 147:923 (1963)). Albumin, insulin and transferrin are provided as a replacement for the need for serum. Since these proteins are of human origin, X Vivo-15 is suitable for growing human cells intended to be injected into autologous recipients.

Of several serum free media tested, medium X Vivo-15 was clearly superior to medium 199 plus 7% FCS for supporting stealth viral growth. The CPE developed more rapidly and there was less of a general loss of vitality seen with the usual stealth viral cultures. Medium X Vivo-15 was also found to be superior to other serum free media, for example, Aims-V medium from GIBCO BRL, Gaithersburg, Md. Aims-V medium also uses human albumin, insulin and transferrin, but the basic salt component is F-10 medium (Ham, Exp. Cell Res., 29:515 (1963)), rather than Iscove's. X Vivo-15 was also superior to medium 199 plus bovine albumin, insulin and transferrin, with or without FCS.

In another aspect a method of detecting a stealth virus is provided by: (1) co-centrifuging a sample of said virus with a permissive cell line of indicator cells; (2) inoculating the cell mixture into culture vessels; and (3) detecting in vitro a CPE in the permissive cell line.

Pre-culture centrifugation (PCC) of patients' lymphoid cells with indicator fibroblast cells refers to co-centrifugation of patients' or animals' lymphocytes with freshly harvested fibroblasts and replacing the cells back into the original tubes containing the fibroblasts.

In preferred embodiments the method involves adding cytomegalovirus (CMV) supernatant, or viral enhancing medium, to the culture and frequently refeeding the culture medium. Viral enhancing medium (VEM) is derived from the supernatants from actively replicating viruses which are functionally related to the stealth virus. Filtered, boiled medium collected from a CMV positive MRC-5 cell line showing a well defined 2+ CPE was able to supplement the stealth viral growth enhancing activity of medium X Vivo-15 used alone and was designated VEM for “viral enhancing medium”. Titration of this medium showed that 20%-30% was adequate to provide significant growth enhancement. X Vivo-15 medium containing 20% each of both CMV and HHV-6 supernatants was more effective than medium containing 20% or 30% CMV supernatant.

In another aspect, a method of detecting a stealth virus is provided by: (1) inoculating a permissive cell line with a sample of said virus in a culture; (2) adding viral enhancing medium to the culture; and (3) detecting in vitro a CPE in the permissive cell line.

In preferred embodiments the viral enhancing medium contains 30% boiled, filtered products derived from the supernatant of cultures of cytomegalovirus and 70% medium X Vivo-15; and the cell line is maintained in a culture medium, that is frequently replaced.

In another aspect culturing a virus is provided by: (a) cocentrifuging a sample of said virus with a permissive cell line of indicator cells; (b) inoculating the cell mixture into culture vessels; (c) adding viral enhancing medium to the culture; and (d) detecting in vitro a CPE in the permissive cell line.

The PCC step in combination with the use of VEM will also improve the detection of the CPE associated with cytomegalovirus (CMV) and human herpesvirus 6 (HHV-6). The growth of CMV and HHV-6, however, are less dependent on these modifications than that of stealth viruses. Furthermore, frequent refeeding of the cultures is not nearly as important for these viruses as it is for stealth viruses.

In preferred embodiments the virus is a stealth virus, cytomegalovirus, or human herpesvirus-6.

The summary of the invention described in detail above is not intended to limit in any way the scope of the present invention which is defined in the appended claims.

### DETAILED DESCRIPTION OF THE INVENTION

Preferred embodiments of the present invention are described in detail below. However, the following description of the preferred embodiments is not intended to limit in any way the scope of the present invention, which is defined in the appended claims.

Toxin production: infrequently fed fibroblast cultures showing an early cytopathic effect (CPE) from a stealth virus inoculum will often display cells with a reduced overall metabolic activity as seen by a less acidic culture than a similarly maintained culture of fibroblasts not containing the stealth virus. As time progresses, it becomes increasingly difficult to retrieve infectious stealth virus from these cultures. Moreover, supernatants from these cultures can lead to an inhibition in the development and even a suppression of an existing CPE in other stealth viral cultures. A general toxic effect can be seen when normal fibroblasts are exposed to such supernatants.

Methods are readily available to fractionate such supernatants and to test individual fractions for toxic activities on both normal cells and permissively infected stealth virus infected cells. Chemical characterization of the components present in active fractions can fully identify their nature. Recombinant DNA technology, can be used to manufacture such compounds in the absence of stealth virus. Antibodies can be produced against the compounds and used both in vitro and potentially in vivo as a way to prevent stealth virus associated toxicity. Antibodies with anti viral growth inhibitory effects could be used to potentiate the in vitro growth of stealth viruses.

Inhibitor Production: A more restricted production of viral growth inhibitory activity in the apparent absence of significant toxic activity is seen when stealth viruses are added to certain T lymphocyte cell lines. In particular, the T cell lines CEM and MOLT-3 obtainable from American type culture collection do not readily support the productive growth of the stealth virus derived from patient D.W. The cells do, however, show enlargement and clumping and retain a vigorous growth. Supernatants collected from these cells show a significant inhibitory effect on the progression of the CPE in other stealth viral cultures. The method of producing this inhibitor is to culture CEM or MOLT cells in X vivo-15 medium. Stealth virus infected human fibroblasts are collected and virus related by freeze thawing. The material is clarified by centrifugation and the supernatant filtered through a 0.45 micron filter. The filtered material is added to cultures of CEM or MOLT cells. Providing sufficient virus is added, many of the lymphoid cells will show some enlargement and clumping. The affected cells become more adherent to the flask and can be enriched by decanting the less adherent, non-affected cells. The remaining culture continues to flourish with regular changes of the media and sub-culturing of the cells. Infectious virus is not generally recoverable from these cultures in spite of the cellular changes seen. Rather the supernatant shows an inhibitory effect on the other stealth virus cultures. The inhibitory activity in the supernatant of these lymphoid cells exposed to stealth virus has been termed “epione”.

Methods are readily available to fractionate such supernatants and to test individual fractions for the presence of epione which can then be chemically characterized. At present, it is postulated that epione will consist of one or more viral component which has a significant structural difference compared to the corresponding component from in the corresponding herpesvirus from which the stealth virus was derived. Recombinant DNA technology, using cloned stealth virus DNA can be used to manufacture epione in the absence of stealth virus. Epione could potentially be used to treat stealth virus infection in vivo. Anti epione antibodies can be produced and used to detect stealth virus infection both in vitro and in vivo. Anti-epione antibodies could also be used to potentiate the in vitro growth of stealth viruses.

As part of a specificity study, the viral growth inhibitory effect of epione containing supernatant was shown to work on fibroblast cells infected with CMV. An inhibitory effect was also seen on the production of human immunodeficiency virus by cultured lymphoid cells from an HIV infected individual. The results were:

P24 ANTIGEN LEVELS
MEASURED AS OPTICAL DENSITY
USING ABBOTT P24 ASSAY

| | Day 5 of Culture | Day 9 of Culture |
|---|---|---|
| | Undiluted Sup. | Diluted 1:30 |
| Untreated cells | 0.389 | 0.822 |
| Cells exposed to | 0.385 | 0.827 |
| medium from | | |
| uninfected CEM | | |
| cultures | | |
| Cells exposed to | 0.068 | 0.057 |
| medium from stealth | | |
| virus infected CEM | | |
| cultures | | |
| Cells exposed to | 0.436 | 0.907 |
| medium from | | |
| uninfected MOLT-3 | | |
| cultures | | |
| Cells exposed to | 0.158 | 0.144 |
| medium from stealth | | |
| virus infected MOLT | | |
| cultures | | |

The present invention provides several culturing conditions that induce a cytopathic effect in a sample containing a stealth virus. These culturing conditions include using approximately 5%-10% fetal calf serum, refeeding the culture medium every 24 to 72 hours using viral enhancing medium and using preculture centrifugation. These conditions are important for inducing a cytopathic effect. For example, the failure to replace the culture medium every 24 to 72 hours often prevents detection of the cytopathic effect. Another example is provided by the fact that not all serum free medium induce a cytopathic effect. Indeed, the use of a basal medium such as minimal essential medium with 2% fetal calf serum and weekly refeeding of the cultures as is commonly practiced in most clinical virology laboratories will not yield a cytopathic effect with primary isolates of a stealth virus. Thus, it can be seen that only a carefully selected specific set of culturing conditions are capable of inducing a cytopathic effect in a sample containing a stealth virus.

Utility

The present invention provides methods for the detection of a stealth virus. A virus is an infective agent and the stealth virus is associated with disease. The disease may be chronic fatigue syndrome (CFS) or one of several other diseases. Therefore, my invention has broad application to any area in which it is important to detect a stealth virus. Such areas include medical, veterinary, and agricultural diagnostics and industrial and pharmaceutical biological quality control.

Many patients with a stealth viral infection have a stealth virus associated disease. Therefore, the detection of the presence of a stealth virus may allow one to confirm the diagnosis of a particular disease. Thus, those patients may avoid needless treatment, including psychiatric treatment. Indeed, it is useful to detect the stealth virus in order to develop a therapy or treatment for patients with a stealth viral associated disease. For example, in order to develop a vaccine it is useful to first detect the virus.

In contrast, many patients without a stealth viral infection do not have a stealth virus associated disease. Thus, the failure to detect a stealth virus may indicate a psychiatric rather than physiological problem. Therefore, those patients may seek proper treatment, such as psychiatric treatment, rather than pursuing treatment aimed at eliminating or reducing the effects of a stealth virus.

Since the stealth virus is associated with disease, it will often times be useful to detect the virus either in order to eliminate or avoid it. For example, in some circumstances a pure material or sample is desired and it would therefore be useful to detect a stealth virus in the material or sample.

It would be useful to know if a food or beverage contained a stealth virus. One could simply avoid consuming the food or beverage in that case as a matter of prudence in order to reduce the risk of contracting a stealth virus associated disease. Similarly, it would be useful to know if a sample of blood contained a stealth virus. One could then simply avoid that blood in order to reduce the risk of contracting a stealth virus associated disease. Many other potential sources of infection are identified herein. Thus, detecting the virus may allow individuals to avoid the virus and any diseases associated with the virus. In other words, the invention is useful for prevention of disease transmission by identifying potential sources of infection.

In another aspect, this invention features diagnostic kits for the detection of Stealth nucleic acid, Stealth antigen, or Stealth antibody in a sample. In a preferred embodiment, the kit has a container containing a nucleic acid probe capable of specifically hybridizing to an Stealth nucleic acid. In another preferred embodiment, the kit has a container containing an antibody capable of specifically binding an Stealth antigen. In another preferred embodiment, the kit has a container for ELISA detection containing a multiwell plates coated with Stealth and ELISA materials for enzyme detection. The ELISA materials contain normal goat serum and peroxidase, labeled goat antihuman IgG and a color change indicator having orthophenylene diamine and hydrogen peroxide in phosphate citrate buffer. Stealth may be lysed before applied to the multiwell plates. In another preferred embodiment, the kit has a container for Western blot detection containing a nitrocellulose sheet, a polyacrylamide slab gel and sodium dodecylsulfate, additionally surfactants as well as pH modifiers and bovine serum albumin and the Fab fragment of normal human IgG, and Western blot analysis container which contains a supply of dilute goat serum and I¹²⁵ labeled goat antihuman immunoglobulin and a source of Stealth. In another preferred embodiment, the kit has a container for the indirect immunofluorescence assay containing human test serum containing Stealth, phosphate buffered saline, and fluorescein-conjugated goat antiserum IgG.

V. Diagnostic Methods and Kits

The present invention provides methods for diagnosing Stealth infection associated with disease by detection of the presence of Stealth in humans and in animals. Both in vitro and in vivo assays can be used, and include, but are not limited to, the culture and molecular probe based assays. In addition, imaging techniques can be used, in which an antibody of the invention or derivative or fragment thereof is bound to a label. The labeled antibody can then be administered in vivo to determine the location of Stealth. In one embodiment, a magnetic resonance imaging agent may be used. In another embodiment, a radioactive imaging agent may be used.

Normal sera or sera from a patient may be used to detect Stealth antigen. Patient sera or normal sera can be used in immunofluorescence assay to detect viral antigens in cells. Preferably, specificity of the reaction can be demonstrated by pre-adsorbing the antibody with supernatant from a virus culture, this blocking specific binding. Non-adsorbed sera will decorate virally infected cells, while absorbed sera will not.

Other immunoassay techniques are also available according to the present invention. These techniques include but are not limited to competitive and non-competitive assays using techniques such as ELISA (Enzyme-Linked ImmunoSorbent Assay), “sandwich” immunoassays, radioimmunoassay, immunoradiometric assay, in situ immunoassays (using colloidal gold, enzyme or radioisotope labels, for example), Western blots, precipitation reactions, gel agglutination assays, hemagglutination assays, complement fixation assays, immunoelectrophoresis assays, to name but a few. In one embodiment, antibody binding is detected by detecting binding of a secondary antibody or reagent to the primary antibody. In a further embodiment, the secondary antibody is labeled. Many means are known in the art for detecting binding in an immunoassay and are within the scope of the present invention.

In a specific embodiment, based on genetic sequence data and identification of open reading frames, synthetic peptides corresponding to the deduced primary amino acid sequence of Stealth proteins can be produced. Peptides or proteins can also be produced by expression of viral genes. In one instance, these genes are expressed by Stealth-infected permissive cells, such as fibroblasts. In another embodiment, a recombinant method of production using a host cell transfected with a viral gene may be used to express the gene. These peptides and proteins can be used to detect antibody in patient sera specific for that antigen. Alternatively, the peptides and proteins can be used as immunogens to prepare anti-Stealth antibody.

In particular embodiments of the invention, the presence of Stealth may be diagnosed by detecting the immunospecific binding of an antibody, or derivative or fragment thereof, reactive with an epitope of an Stealth in a sample. In another embodiment, hybridization of a nucleic acid probe is detected.

The sample from a subject (i.e., a patient, an animal, or a cell culture) may consist of any body fluid, including but not limited to peripheral blood, plasma, cerebrospinal fluid, urine, throat swabs, surgically excised and fine needle aspirated tissue samples, other body fluids. Preferably the sample is blood, CFS, or affected tissue, i.e., brain biopsy. Binding of an antibody or a molecular probe may be accomplished and/or detected in vitro or in vivo. In vitro binding may be performed using histolic specimens or subfractions of tissue or fluid. In vivo binding may be achieved by administering the molecular hybridization (nucleic acid) probe or antibody (or fragment or derivative thereof) by any means known in the art (including but not limited to intravenous, intraperitoneal, intranasal, and intraarterial, to name but a few) such that specific binding may be detected; for example, by attaching a radioactive label or diagnostic antibody, fragment, or derivative or by attaching such a label to its specific binding partner.

It should be understood that the diagnostic methods of the invention are best used in the context of other diagnostic parameters in order to obtain a comprehensive patient diagnosis. Clinical features of psychiatric, neurological, rheumatological, auto-immune, and other diseases may accompany Stealth infections in different patients.

As with any diagnostic criteria, the parameters disclosed in the present invention may not be sole determinants, or pathognomonic, of a particular disorder.

According to the invention, Stealth infection may be diagnosed and monitored by detecting the presence of nucleic acid sequences homologous to an Stealth gene from a sample. Several procedures could be used to correlate Stealth genes or gene expression with disease. These involve (1) producing and analyzing cDNA or genomic DNA libraries obtained from a patient suffering from the disease to determine the presence of Stealth genes; (2) analyzing disease samples by Southern blot to determine whether specific genetic polymorphisms, RFLPs) or oligoclonal Stealth sequences exist; (3) analyzing disease samples by the polymerase chain reaction (PCR) amplification, and analysis of PCR products using electrophoresis and/or hybridization procedures. The PCR procedure represents a more efficient procedure in the time required for analysis and in the number of patients that can be analyzed to detect a disease correlation. A fourth procedure using in situ hybridization of cells may also be extremely useful.

A kit for detecting the presence of Stealth in a sample is also provided. Such a kit comprises, in a suitable container, an Stealth-specific probe. In one embodiment, the probe is an antibody specific for an Stealth antigen. In another embodiment, the probe is a nucleic acid (molecular probe) capable of hybridizing to an Stealth nucleic acid sequences.

Antibodies and polypeptides prepared as described above can be used in various immunological techniques for detecting Stealth and anti-Stealth antibodies in physiological specimens, particularly body fluid samples, including blood, plasma, serum, urine, and the like, and cell samples, such as lymphocytes. Depending on the nature of the sample, both immunoassays and immunohistochemical staining techniques may find use.

Liquid phase immunoassays and Western blot analysis will find use in detection of Stealth in body fluids, particularly blood and urine. The use of antibodies in protein binding assays is well established. Numerous competitive and noncompetitive protein binding assays have been described in the scientific and patent literature, and a large number of such assays are commercially available.

Radioimmunoassay techniques for detecting antibodies in a physiological specimen of susceptible patients include radiolabeled assays of the so-called blot technique, such as the Western Blot technique. Also operable and most preferred for the detection of antibodies are the enzyme-linked immunosorbent assay (ELISA). ELISA and Western Blot techniques start with the virus. Thirdly, antibodies to Stealth may be detected by an indirect immunofluorescence assay. This assay is significant because it uses the infected T-cell as a starting material.

A. ELISA

In general, the ELISA technique involves reacting a lysate of Stealth with a test specimen taken from a patient. The mixture is incubated with a peroxidase labeled antibody in wells. Any well positive for the presence of Stealth antibodies forms a detectable and measurable color product.

Sodium dodecylsulfate (0.1% SDS)-disrupted purified Stealth are diluted in coating buffer (0.1M NaHCO₃, pH 9.6) to 2 μg/ml, and 0.1 ml (200 ng) of this viral suspension is added to each well of the Immulon II microtiter plates. The plates are incubated for 12 to 16 hrs at 37° C., and then washed with wash solution (0.05%-Tween-20, 0.15M NaCl). The test sera are diluted 1:100 in Buffer 3 (0.15M NaCl, 1 mM edetic acid, 0.05% Tween-20, 0.1% bovine serum albumin, pH 7.4), and 0.1 ml of the diluted sera are added to each well in duplicate and incubated for 60 min. at 37° C. The plates are then washed and incubated with 1:100 dilution of peroxidase-conjugated rabbit anti-cat IgG (Cappel Laboratories, Conchranville, Pa.) for 30 min at 37° C. After another wash cycle, the plates are incubated with a fresh substrate solution (50 mM citric acid adjusted to pH 4.0, 0.2 mM 2, 2′-azinobis-3-ethylbenzthiazoline-sulfonic acid, 2 mM H₂O₂) for 10 min at room temperature. The reaction is stopped with the addition of 0.1 ml of 0.2M hydrogen fluoride and the absorbance at 405 nm is measured by an ELISA plate reader. Each ELISA plate contains duplicate wells of positive and negative control sera which are used as internal standards for diagnostic comparison.

In another embodiment, the wells of a 96-well plate are coated overnight with a lysate of Stealth at 0.5 μg protein per well in 100 μl 50 mM sodium bicarbonate buffer, pH 9.6. The wells are washed with water and incubated for 20 minutes with 100 μl of 5% bovine serum albumin in phosphate buffered saline (PBS). After washing, 100 μl of 20% normal goat serum in PBS are added to each well, followed by 5 or 10 μl of the test specimen, and allowed to react for 2 hours at room temperature. The wells are washed three times with 0.5% Tween-20 in PBS in order to remove unbound antibodies and incubated for 1 hour at room temperature with peroxidase labeled goat anti-human IgG at a dilution of 1:2000 in 1% normal goat serum in PBS. Goat anti-human IgG is a second antibody that binds with the antibody antigen complex formed in positive wells. The wells are successively washed 4 times with 0.05% Tween 20 in PBS and 4 times with PBS to remove unbound goat antibody and reacted with 100 μl of the substrate mixture containing 0.05% orthophenylene diamine and 0.005% hydrogen peroxide in phosphate-citrate buffer, pH 5. This substrate mixture detects the peroxidase label and forms a color product. The reactions are stopped by the addition of 50 μl of (NH4)2SO4 and the color yield measured using an ELISA reader which quantifies the color reading. Assays are performed in duplicate; absorbance readings greater than three times the average of 4 normal negative control readings are taken as positive.

B. Western Blot

As indicated above, antibodies to Stealth may also be detected in a physiological specimen of susceptible patients by means of the Western Blot technique. Stealth is lysed and electrophoretically fractionated on a polyacrylamide slab gel. Protein bands on the gel are then electrophoretically transferred to a nitrocellulose sheet according to the procedure of Towbin et al., Proc. Natl. Acad. Sci. USA, 76:4350 (1979). The sheet is incubated at 37° C. for 2 hr. with 5% bovine serum albumin in 10 mM Tris-HCl, pH 7.5 containing 0.9% NaCl and cut into 0.5 cm strips. Each strip is incubated for 2 hr. at 37° C. and 2 hours at room temperature in a screw cap tube containing 2.5 ml of buffer 1 (20 mM Tris-HCl, pH 7.5, 1 mM EDTA, 0.2 M NaCl, 0.3w Triton X-100 and 2 mg/ml bovine serum albumin and 0.2 mg/ml of human antibody fractions, Fab). Test specimen are then added to individual tubes containing the strips and incubation continued for 1 hour at room temperature and overnight in the cold. The strips are washed three times with solution containing 0.5% sodium deoxycholate, 0.1 M NaCl, 0.5% Triton X-100, 1 mM phenylmethylsulfonyl fluoride and 10 mM sodium phosphate, pH 7.5. The strips are incubated for 1 hour at room temperature with 2.4 ml of buffer 1 and 0.1 ml of normal goat serum. Affinity purified and ¹²⁵I-labeled goat anti-human immunoglobulin (mu chain and Fc fragment) (1.25×10⁶ cpm) are added to the reaction mixture and the incubation continues for 30 min. at room temperature. The strips are washed as described, dried, mounted and exposed to X-ray film.

In another embodiment, sucrose-gradient purified virus is disrupted with 0.1% SDS and electrophoresed on a 8% preparative polyacrylamide slab gel (with 5% stacking gel) in presence of 0.1% sodium dodecyl sulfate (SDS). The separated viral proteins are transferred electrophoretically onto a nitrocellulose filter which is then treated with Buffer 3 containing 3% gelatin and cut into strips of identical size. Two lanes of each gel containing molecular weight standards are stained with amido black and later used for molecular weight comparison of the immunoblots.

Serum samples are diluted to 1:100 in Buffer 3 and incubated with blot strips in separate glass tubes for 60 min at 37° C. The blot strips are washed individually and incubated with peroxidase-conjugated rabbit anti-cat IgG (Cappel Laboratories) at a dilution of 1:400 for 60 min at 37° C. After extensive washing, each strip is incubated with a fresh substrate solution (0.05% diaminobenzidine and 0.01% H₂O₂ in 0.1M Tris, pH 7.4) for 4-10 min at room temperature. The reaction is stopped with excess distilled H₂O upon establishment of visible bands, and the strips are blot dried. The molecular weights of the bands on the immunoblots are then determined by comparing the migration distance with molecular standards from the original gel. Positive and negative control sera are included in each Western blot analysis as internal control for diagnostic evaluation.

C. Indirect Immunofluorescence Assay for Antibodies to Stealth

Infected cells are washed with phosphate buffered saline (PBS) and resuspended in PBS at 10⁶ cells/ml. Approximately 50 samples of cell suspension are spotted on a slide, air dried, and fixed in acetone for 10 min. at room temperature. Slides are stored at −20° C. until ready for use. 20 ml of the test specimen diluted 1:10 in PBS are added to the fixed cells and incubated for 1 hr. at 37° C. Slides are washed and reacted for 30 min. at room temperature with a dilute solution of fluorescein-conjugated goat anti-human IgG. Slides are again washed and examined under a microscope for fluorescence. A negative control uses uninfected parental cells. The above describes a fixed cell system in which the antibody-antigen reaction is sought for both inside and outside the cell.

For live cell immunofluorescence assay all the above reactions are done in a tube instead of on a slide, but without chemical fixation of the cells. After reaction with the fluorescein conjugated antihuman antibody, the cells are added to the slide and examined under a microscope for antibody-antigen reaction on the surface of the cell.

D. Test Kits

Stealth test kits are constructed for detecting antibodies using several different techniques for detection. One test kit for antibodies detection comprises a compartmented enclosure containing a plurality of wells, plates which are coated prior to use with Stealth and ELISA materials for enzyme detection consisting of normal goat serum and peroxidase, labeled goat antihuman IgG, and a color change indicator consisting of orthophenylene diamine and hydrogen peroxide in phosphate citrate buffer.

A second test kit for detecting antibodies using the Western Blot technique comprises a container containing a nitrocellulose sheet and a polyacrylamide slab gel in the presence of sodium dodecylsulfate, and additionally surfactants as well as pH modifiers and bovine serum albumin and human Fab. Additionally this Western Blot analysis container also contains a supply of dilute normal goat serum and ¹²⁵I labeled goat antihuman immunoglobulin and a source of Stealth.

Finally, an Stealth specific test kit for detecting antibodies using the indirect immunofluorescence assay comprises a compartmental container, human test serum containing Stealth, phosphate buffered saline, and a fluorescein-conjugated goat antiserum IgG.

E. Polynucleotide Probes

Diagnostic tests for detecting the presence of Stealth in biological samples may also be performed using polynucleotide probes. Such polynucleotide probes may be prepared based on the sequence of the viral genome. The length of the probe is not critical, but will usually comprise at least about 12 bases, more usually comprising at least about 16 bases, which are substantially complementary to a portion of the viral genome. The probe itself may be DNA, RNA, or their analogs, and the probe need not have perfect match with the Stealth genome. One or two mismatched pairs are acceptable for probes up to 20 bases in length and three to five mismatched pairs in probes from 20 to 35 bases. The probes may be prepared synthetically to include a detectable label. Usually, the synthetic sequences are multiplied in commonly available cloning vectors and suitable hosts or by PCR in order to obtain large quantities. The vectors may themselves be labelled for use as probes, or shorter fragments containing complementary strands may be excised and labelled. Methods for the preparation and utilization of nucleotide probes for diagnostic testing are described in Sambrook et al., the disclosure of which is incorporated herein by reference.

A variety of labels may be employed, including those which have been described above for use in immunoassay, particularly radionucleotides. Suitable labels may be bound to the probe by a variety of techniques. Commonly employed is nick translation with α-³²P-dNTP terminal phosphate hydrolysis with alkaline phosphatase followed by 5′-end labelling with radioactive ³²P employing λ-³²P-NTP and T4 polynucleotide kinase or 3′-end labelling with an α-³²P-dNTP and terminal deoxynucleotidyl transferase. Alternatively, nucleotides can be synthesized where one or more of the atoms present are replaced with a radioactive isotope, e.g., hydrogen with tritium. In addition, various linking groups can be employed. The terminal hydroxyl can be esterified with inorganic acids, e.g., ³²P phosphate or ¹⁴C organic acids, or else esterified with bifunctional reagents to provide other reactive groups to which labels can be linked.

The present invention is also useful in a variety of other ways readily apparent to those skilled in the art.

### EXAMPLES

This invention will be more fully understood with reference to the examples which follow. The following examples are intended to illustrate the invention, but not to limit its scope which is defined in the claims appended hereto. The following examples are presented to illustrate the advantages of the present invention and to assist one of ordinary skill in the art in making and using the same, but are not intended in any way to otherwise limit the scope of the disclosure or the protection granted by a letters patent hereon.

In the following examples, the detection of the cytopathic effect was recorded as follows. This minimal level of CPE has to be transferable to secondary cultures for the culture to be recorded as a true positive (+). A + (one plus) positive effect indicates the presence of multiple enlarged retractable cells forming small clumps with indistinct cell borders are clearly seen in the cultures. A +/− (plus/minus) or equivocal effect refers to a minimal change in the culture in which the rounded cells are either not enlarged or non-refractile (possibly dead). Less than 4% of cultures from CFS patients have been recorded as equivocal, either because the CPE has been restricted to only a small region of the culture, or because it has failed to recur on secondary passage. These cultures have been excluded from analysis.

A ++ (two plus) strong positive response is recorded when vacuoles are clearly identified within enlarged, rounded, retractile cells and/or when multiple foci of cell clumping and/or destruction are apparent which are ringed by cells described as having a positive effect. A +++ (three plus) very strong positive effect refers to extensive ++ cellular changes throughout much of the culture and/or the appearance of large refractile vacuolated, foamy syncytial cell formation. Typical CPE's are shown in Patent Cooperation Treaty publication number WO 92/20787.

A benefit of using multiple indicator cell lines, is that the CPE appearances in the different cell lines sometime complement each other. For example, cell syncytia are usually best observed in monkey kidney cells. The smaller rounded retractile cells are usually best observed in the human fibroblasts. In over 80% of positive cultures, the CPE is clearly observable in at least 2 of the 3 cell lines. As with any viral induced CPE, it is important to confirm transmission to fresh cultures. In recipient cultures, the stealth virus induced CPE generally develops more rapidly than in the primary culture with at least the same level of intensity.

Although the appearances of the CPE share some features in common with those associated with cytomegalovirus (CMV) and herpes simplex virus (HSV) infection, stealth virus cultures can be readily distinguished from cultures harboring these viruses. The CPE from HSV is much more rapid, occurring in a matter of days. The destruction is greater with large masses of dead cells. The CPE from CMV tends to initially develop in smaller, looser clusters and evolves more slowly than observed with stealth viruses. The intracytoplasmic vacuolization and syncytia formation are far less prominent with CMV than with stealth viruses. The stealth virus infected cells give more the appearance of “foamy cells” than does CMV. Indeed, with some stealth viral cultures, there appears to be a marked accumulation of lipid-like material attaching itself to the wall of the culture tube. Human CMV does not infect monkey derived cells.

### Example 1

### Culture of Stealth Viruses—7% FCS and Frequent Refeeding

Culture tubes containing human fibroblast (MRC-5), primary human foreskin fibroblast (MRHF) and rhesus monkey kidney (RMK) cells are each inoculated with a cellular mixture of the buffy coat granulocytes and ficoll-hypaque separated lymphocytes derived from approximately the equivalent of one milliliter (ml) of heparinized blood. Typically, 5 ml of blood collected into a “green top” heparinized tube, are layered onto 3 mls of a ficoll-hypaque lymphocyte separation medium. Following 20 minutes centrifugation at 1,500 rpm, the lymphocyte, which collect at the plasma:ficoll hypaque interface, and the “buffy coat”, present on the top of the erythrocyte layer, are collected into approximately 1 ml. An effort is made to minimize the number of erythrocytes in collecting all of the visible buffy coat.

The cell mixture is washed once in 1 ml of 199 medium containing 7% FCS and resuspended into 1 ml. Aliquots of 0.2 ml of the cells are added to culture tubes containing 1 ml of 199 medium plus FCS. The tubes are placed in an incubator at 37° C. for 45-60 minutes. The are then rinsed to remove macroscopically visible erythrocytes and other non-adherent cells. Rinsing (washing) consists of emptying the fluid content of the culture tube by decanting or by aspiration; adding 2 mls of medium or phosphate buffer saline; rocking the tube for several seconds to suspend erythrocytes; decanting the tube again. This important step is performed 2-10 times or until there are no macroscopically visible erythrocytes.

Two mls of medium with 7% FCS, are added and the tubes returned to a 37° C. incubator. Cultures are maintained in the incubator at 37° C., with refeeding (replacement of old medium with medium) at 24, 48 and 72 hours. The tubes are examined microscopically after the 24 hour refeeding and, if residual erythrocytes are present, the tubes are rinsed in a manner similar to that performed at the 45-60 min. step. The tubes are refed three times each week by replacing the old medium with 2 mls of fresh medium. This procedure is designed to reduce the accumulation of a toxic component in the culture medium which tends to suppress viral growth. The tubes are examined three times per week for evidence of CPE (CPE). CPE is generally recognizable between 2-3 weeks after culturing.

A lack of regular refeeding of the cultures can result in a tendency for the CPE to abort and not to progress. This effect is not seen with CMV infected cultures. In unfed stealth virus cultures and even with regular refeeding, one can observe a toxic effect on many of the remaining cells. Culture cells appear to lose a degree of vitality and become duller in appearance compared to control cultures. The fibroblasts can assume somewhat of a pavement appearance, instead of the elongated shape. Some of these changes can be mimicked using 5 nM of the polyether marine toxin okadaic acid (Cohen et al., “Okadaic acid: A new probe for the study of cellular regulation” Trends Biochem. Sci., 15:98-102 (1990). CMV positive cultures do not demonstrate the toxic activity such as that observed with stealth viruses.

Moreover, the detection of CPE from CMV is readily seen in cultures containing only minimal essential medium and 2% FCS even without regular refeeding of cultures. This is the routine medium used in most virology laboratories and can be contrasted with the more enriched medium 199 and 7% FCS that is used to culture stealth viruses. The more enriched medium and the higher concentration of FCS, help to neutralize the toxic, stealth virus growth inhibiting effects, which would otherwise occur in the cultures.

Although presumptive of stealth virus infection, the CPE appearance may require additional confirmation for a definitive conclusion of stealth virus infection.

### Example 2

### Viral Enhancing Medium (VEM)

In preparing viral enhancing medium a known positive CMV culture was passaged into a flask of MRC-5 cells and fed with X Vivo-15 medium. The cultures were observed for the development of CPE. The culture medium was changed at 1 week when approximately 50% of the cells showed signs of CPE, but before there was marked cellular destruction. This newly added medium was collected 48 hours later. It was centrifuged at 800 g for 20 minutes to remove cellular debris.

The medium was transferred to new tubes which were placed in a beaker containing water. The water was heated to boiling for 20 minutes. After cooling, the medium was filtered through a 0.45 micron Millipore filter. This material was diluted 30:70 into regular X Vivo-15 medium to constitute a “lot” of CMV derived VEM. Each lot of VEM is tested to confirm: i) that it does not contain any residual live CMV by adding the medium to MRC-5 cells; and ii) that it can promote the development of the CPE induced by the prototype stealth virus by comparing the growth of the stealth virus in RMK cells containing VEM with that of similarly inoculated cells containing X Vivo-15 medium without supplement.

VEM has been tested on ten additional stealth viral isolates and clearly enhanced the growth of all of them. Two of these isolates are known to share CMV related sequences with the prototype stealth virus. Another isolate (from patient G.P.) shares antigens with HHV-6, rather than CMV, and is considered an HHV-6 related stealth virus. Another isolate appears to have an adenoviral sequence. The use of VEM also reduced the time for a discernible CPE using fresh blood from two newly cultured CFS patients. It enhances the intensity of the CPE and reduces the tendency for weekly positive cultures to revert to near normal appearance. VEM has also worked well in the cultures from the tissues of cats inoculated with the prototype stealth virus from patient D.W. allowing for clearly positive culture results. VEM obtained from HHV-6 (strain GS) infected fibroblasts was similarly tested for its ability to promote the growth of CMV and HHV-6 associated stealth viruses. It worked well with both viral types with a discernable advantage on the HHV-6 related stealth virus from patient G.P., compared to the CMV related stealth virus from patient D.W.

As a specific example, cultivation of a prototype stealth virus isolated from a CFS patient (initials D.W.) can be greatly enhanced by the addition to the culture of VEM comprising a 30% concentration of boiled, filtered supernatants from cytomegalovirus (CMV) infected cultures. This addition helps remedy a deficiency of viral growth enhancing components coded for by the immediate-early (I-E) and probably other CMV related genes which are not detectable in the stealth virus from this patient.

### Example 3

### Viral Enhancing Medium and Pre-Culture Centrifugation

Human fibroblast (MRC-5), rhesus monkey kidney (RMK) and rabbit kidney (RK) cell lines were obtained from BioWhittaker, Inc., Walkersville, Md. The tubes were placed in a 370 incubator. The next day, the Delbecco's modified Eagles medium containing 2% FCS is replaced by medium 199 plus 7% FCS. The tubes were used to provide indicator cells for stealth viral cultures within the next 7 days. To establish the viral cultures, the contents of a single test tube of each of the indicator cell lines to be used were scraped from the tubes and washed once in X Vivo-15 medium.

The cells were gently resuspended into approximately 0.5 ml of medium and transferred to 2 ml Eppendorf tubes. Prior to this step, ficoll-hypaque separated lymphocytes from either heparinized or citrate treated whole blood, were obtained by layering 5 mls of anti-coagulated blood onto 3 mls of ficoll-hypaque solution in 12 ml conical tubes. The tubes were centrifuged for 20 min at 800 g. The banded lymphocytes were aspirated and transferred to a fresh tube for washing in 10 mls of medium. The lymphocytes were resuspended in approximately 1 ml. Aliquots of 0.2 ml of the lymphocytes were added to each of the Eppendorf tubes containing the harvested fibroblast indicator cells with a final aliquot stored for future studies.

The lymphocyte-fibroblast cell mixture was centrifuged at high speed for 3 minutes. The tightly-packed cell pellet was gently resuspended and transferred back to the tube from which the fibroblasts were originally obtained. Two mls of VEM (X Vivo-15 medium supplemented with 30% CMV supernatant) were added and the tubes are placed in an incubator at 37° C. The tubes were refed with VEM at 48 and 72 hours and thereafter 3 times per week.

Control cultures in which either lymphocytes from other individuals are used, or the fibroblasts were processed but with the exception of no added lymphocytes, were fed in parallel with the test cultures. Note, in this revised protocol, buffy coat granulocytes are no longer routinely used since the contaminating erythrocytes tended to clump about the fibroblasts during the centrifugation step and were difficult to remove in subsequent washing of the cultures. Granulocytes may be an important source of virus in some patients. If this proves to be so, leucocyte rich plasma will be obtained by dextran precipitation of the erythrocytes from anti-coagulated blood, or as an alternative, modifications of the ficoll-hypaque separation method can be used which will separate both lymphocytes and granulocytes away from the erythrocytes. For example by using PMN isolation medium from Robbins Scientific Corp., Sunnyvale Calif. CSF and tissue extracts can be used in place of the lymphocytes.

The cultures were observed for a CPE which characteristically consists of rounding and swelling of the cells, formation of cell clumps which tend to disperse, appearance of intracellular granules/vacuoles and an overall foamy cell appearance often with prominent accumulation of lipid-like material.

TABLE 3
Examples of the Enhanced Recovery and More Intense
Development of CPE by a Stealth Virus from a CFS Patient
Using Pre-Culture Centrifugation (PCC) and Viral Enhancing
Medium (VEM).*

| Method of Culturing | Time to CPE** | Intensity of CPE |
|---|---|---|
| Patient 1 | | |
| Medium 199 + 7% FCS | 45 days | 1-2+ |
| PCC and VEM | 12 days | 3+ |
| Patient 2 | | |
| Medium 199 + 7% FCS | 28 days | 1-2+ |
| PCC and VEM | 16 days | 3+ |

*Medium X Vivo-15 containing 30% supplement of boiled filtered supernatant from a CMV culture also grown in medium X Vivo-15.
**Results are in RMK cells. Enhanced growth was also seen in MRC-5 cells.

10. Growth of Stealth Virus in Insect Cell Line

The Spodoptera fruiperda derived ovarian cell line Sf9 that is used routinely for the propagation of recombinant insect baculovirus was obtained from PharMigen, San Diego. It was maintained at 27° C. in Grace's medium with 10% fetal calf serum. The stealth viruses from patients D.W., G.P., K.E. and B.B. were passaged into the insect cell line using 0.1 ml of cell-free supernatant from an infected MRC-5 human fibroblast culture. CPE was clearly seen within two days and progressed over the next several days.

The infected cultures showed enlarged foamy cell syncytia. Virus infectious for MRC-5 and for insect cell cultures was recoverable from the insect cell cultures to a dilution of 10⁻³ ml. Electron microscopic examination of the insect cultures infected with the virus from patient D.W. showed abundant herpes-like viral particles. In control studies, neither cytomegalovirus, human herpes virus 6, varicella zoster virus or Epstein-Barr virus induced CPE in the insect cell line; nor was infectious virus recoverable from these cultures.

Deposit of Microorganisms

The stealth virus isolated from patient D.W. (virus-X infected MRC-5 cells) was deposited with the American Type Culture Collection (ATCC) -12301 Parklawn Drive, Rockville, Md. 20852, under the provisions of the Budapest Treaty on the International Recognition of the Deposit of Microorganisms for the Purposes of Patent Procedures on 9-17-91, and were assigned accession no. VR-2343.

The present invention is not to be limited in scope by the microorganisms deposited or the specific embodiments described herein since such embodiments are intended as but single illustrations of one aspect of the invention and any microorganisms which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims.

Various references are cited herein, the disclosures of which are incorporated by reference herein in their entirety. In particular Martin, et al. The American Journal of Pathology, 145(2): 440-451, 1994 and Appendix A attached hereto as part of the specification are both incorporated herein by reference in their entirety, including any drawings.

What is claimed is:

1. A kit for in vitro culture of stealth virus comprising an agent that neutralizes stealth virus-associated toxic activity.

2. The kit of claim 1 in which the neutralizing agent is an antibody to the stealth virus-associated toxin.

3. The kit of claim 1 wherein said kit contains viral enhancing medium.

4. The kit of claim 1 further comprising a package insert.

5. The kit of claim 1 further comprising an apparatus with a means for detecting a cytopathic effect.

6. The kit of claim 1 further comprising an apparatus with a means for detecting a stealth viral antigen.

7. The kit of claim 1 further comprising an apparatus with a means for detecting a stealth viral nucleic acid.

8. A diagnostic kit for detecting the presence of Stealth Virus in a sample comprising a container containing a nucleic acid probe capable of specific hybridization to a Stealth Virus nucleic acid.

9. A diagnostic kit for detecting the presence of a Stealth Virus in a sample comprising a container containing an antibody capable of immunospecifically binding a Stealth Virus antigen.

10. A diagnostic test kit for detection of Stealth Virus specific antibodies in a sample using the ELISA technique comprising a compartmented enclosure containing multiwell plates which are coated with said virus and ELISA materials for enzyme detection.

11. A diagnostic test kit for detection of Stealth Virus specific antibodies in a sample using the Western Blot technique, comprising a container, a cover, wherein said container contains
a nitrocellulose sheet and a polyacrylamide slab gel and sodium dodecylsulfate,
additionally surfactants as well as pH modifiers and bovine serum albumin and the Fab fragment of normal human IgG, and
Western Blot analysis container which contains a supply of dilute normal goat serum and ¹²⁵I labeled goat antihuman immunoglobulin and a source of Stealth Virus.

12. A diagnostic test kit for detection of Stealth Virus specific antibodies in a sample using the indirect immunofluorescence assay, comprising a container, human test serum containing Stealth Virus, phosphate buffered saline, and fluorescein-conjugated goat antiserum IgG.
